(19) United States
(12) Patent Application Publication
(10) Pub. No.: US 2010/0019125 A1
(43) Pub. Date: Jan. 28, 2010

(54) System and method for laser calibration

(76) Inventors: Mario Antonio Stefani, Sao Paulo (BR); Jarbas Caiado de Castro Neto, Sao paulo (BR); Alessandro Damiani Mota, Sao Paulo (BR); Giuliano Rossi, Sao Paulo (BR)

Correspondence Address: JONES DAY, 222 EAST 41ST ST, NEW YORK, NY, 10017, US

(21) Appl. No.: 11/921,115

(22) PCT Filed: May 29, 2006
(86) PCT No.: PCT/AU2006/000721

§ 371 Date: Aug. 26, 2009

(30) Foreign Application Priority Data
May 27, 2005 (AU) 2005902720

(51) Int. Cl. A61B 18/20

(52) U.S. Cl. 250/205

(57) ABSTRACT

A laser calibration system and method are described for a laser unit (10) operable to fire a laser beam that is guided along an optical delivery path (310) to a delivery point at a distal end of the optical delivery path. The laser calibration system comprises a laser controller operable to drive the laser unit (10) to fire the laser beam dependent on a desired laser power and a compensation factor associated with the optical delivery path. A detector (70) generates a measurement signal related to laser power at the delivery point; and a laser calibrator (802) generates an error signal dependent on a comparison (810) of the desired laser power and the measurement signal and to adjust the compensation factor dependent on the error signal.

### FIELD OF THE INVENTION

This invention relates to the calibration of lasers and, in particular to the calibration of a laser system having an associated optical delivery path to provide a laser beam for treatment of a patient's retina.

### BACKGROUND OF THE INVENTION

Lasers have found significant use in medical procedures, including surgery, tattoo removal and optical applications.

For example, in a procedure described in WO 02/094260, the contents of which are herein incorporated by reference, a 810 nm ophthalmic laser is used to carry out the so-called Indocyanine Green-Mediated Photothrombosis (i-MP) procedure on a patient's eye. This requires a very precise delivery of output power from the laser beam to the eye. If the output power has greater than 5% deviation from the desired output power required to treat the eye, this can lead to insufficient or over-exposure and can thereby negate the therapeutic effects of the procedure.

Lasers used to treat the target tissue are therefore often equipped with a feedback device referred to as a “power monitor” to address any deviations of the output power. Further cut-out safety mechanisms may ensure that the laser power remains within a range deemed safe for treatment of humans.

Components of the laser console are generally calibrated at the factory prior to sale. With time these components can become un-calibrated and further exacerbate inaccuracies of output power from the laser console itself. Such components include the laser source, sensory photodiodes and other power monitor components which monitor the output power of the laser source. Power deviations caused by these components are detected by the power monitor within the laser console, but since the tolerance levels are set to accuracies of ±20% in accordance with the International Electrotechnical Commission (IEC) requirements, the power deviation of the laser beam that is delivered to the patient's eye may be well beyond minimum safety levels for some procedures such as i-MP.

In a typical procedure to treat a patient's eye (such as that described in WO 02/094260), a number of additional components are placed in the path of the laser beam between the laser and the patient's eye.

Referring to FIG. 1, once the laser beam exits the laser console 10 the laser beam travels through an optical delivery system. The optical delivery system includes, for example, optical fibre leading to a slit lamp adaptor 30 that, in turn, is attached to a slit lamp microscope 40 and a laser contact lens 60. The slit lamp adaptor 30 is a standard unit which itself includes: a fibre optic cable, a Galileo type microscope (not shown) designed to control the laser beam spot size; a mechanical system (not shown) to attach the device to the slit lamp microscope 40, and a beam splitter 50 to position the laser beam coaxially into the optical path of the slit lamp microscope 40. The slit lamp adapter 30 may be used to adjust the laser spot size dependent on the dimensions of features of the eye 100 which are to be examined or treated.

In addition and as shown in FIG. 1, another optical component commonly known as a laser contact lens 60 is placed in the optical delivery path by the ophthalmologist, and is used to enhance the visualisation of the retina architecture of the patient's eye 100.

All of these components in the optical delivery path may cause further uncontrolled and unpredictable output power losses to the resulting laser beam which actually contacts the eye. These losses can be caused by factors such as:
Accumulation of dust or dirt on the optics of the beam splitter, fibre optic tip, objective lens and contact lens;
Degradation or ‘wear and tear’ of the fibre optic;
Micro fissures in the fibre optic;
Misalignment of fibre optic couplings; and
Ageing of the laser diode.

These losses are not accounted for in the standard power control and monitoring functions within the laser console 10 of current laser systems. The inventors estimate that the optical delivery path can cause losses of greater than 10%, which is considered above the limits specified by the i-MP Clinical Protocol.

Reference to any background art in the specification is not an acknowledgement or suggestion that this background art forms part of the common general knowledge in Australia or any other jurisdiction or that this background art could reasonably be expected to be ascertained, understood and regarded as relevant by a person skilled in the art.

### SUMMARY OF THE INVENTION

According to a first aspect of the invention there is provided a method of calibrating a laser system in which a laser unit is operable to fire a laser beam that is guided along an optical delivery path to a delivery point at a distal end of the optical delivery path, the method comprising:

defining a desired laser power for the optical delivery path;

initialising a compensation factor for the optical delivery path;

driving the laser unit to fire a laser beam dependent on the desired laser power and the compensation factor;

receiving a measurement signal related to laser power at the delivery point;

comparing the measurement signal and the desired laser power to generate an error signal; and

adjusting the compensation factor dependent on the error signal.

According to a second aspect of the invention there is provided a laser calibration system for a laser unit operable to fire a laser beam that is guided along an optical delivery path to a delivery point at a distal end of the optical delivery path, said laser calibration system comprising:

a laser controller operable to drive the laser unit to fire the laser beam dependent on a desired laser power and a compensation factor associated with the optical delivery path;

a detector operable to generate a measurement signal related to laser power at the delivery point; and

a laser calibrator adapted to generate an error signal dependent on a comparison of the desired laser power and the measurement signal and to adjust the compensation factor dependent on the error signal.

According to a further aspect of the invention there is provided a laser system including:

a laser for generating a beam of laser light;

an optical delivery path provided by at least one selected component for a given procedure;

a detector for placement at the end of the optical delivery path for measuring the power of the laser beam at the end of the delivery path; and

laser power modification means for modifying the power of the laser beam in accordance with the measurements obtained by the detector.

According to a further aspect of the invention there is provided a computer program product comprising machine-readable program code recorded on a machine-readable recording medium, for controlling the operation of a data processing apparatus on which the program code executes to perform a method of calibrating a laser system in which a laser unit is operable to fire a laser beam that is guided along an optical delivery path to a delivery point at a distal end of the optical delivery path, the method comprising:

defining a desired laser power for the optical delivery path;

initialising a calibration factor for the optical delivery path;

driving the laser unit to fire a laser beam dependent on the desired laser power and the calibration factor;

receiving a measurement signal related to laser power at the delivery point;

comparing the measurement signal and the desired laser power to generate an error signal; and

adjusting the calibration factor dependent in the error signal.

According to a further aspect of the invention there is provided a computer program comprising machine-readable code for controlling the operation of a data processing apparatus on which the program code executes to perform a method of calibrating a laser system in which a laser unit is operable to fire a laser beam that is guided along an optical delivery path to a delivery point at a distal end of the optical delivery path, the method comprising:

defining a desired laser power for the optical delivery path;

initialising a calibration factor for the optical delivery path;

driving the laser unit to fire a laser beam dependent on the desired laser power and the calibration factor;

receiving a measurement signal related to laser power at the delivery point;

comparing the measurement signal and the desired laser power to generate an error signal; and

adjusting the calibration factor dependent in the error signal.

### BRIEF DESCRIPTION OF THE DRAWINGS

An embodiment of the invention is now described with reference to the Figures, in which:

FIG. 1 shows a prior art laser system that includes a laser unit and an optical delivery path;

FIG. 2 shows a laser system having a detector positioned in the optical delivery path and providing a feedback signal to calibrate the laser unit;

FIG. 3 is a schematic block diagram showing the laser unit of FIGS. 1 and 2 in greater detail;

FIG. 4 is a functional block diagram of a laser controller for use in the systems described herein;

FIG. 5 shows a functional block diagram of a subsystem of the laser controller of FIG. 4;

FIG. 6 shows a graph of errors due to non-linearity in a laser diode;

FIG. 7 shows a functional block diagram of a laser controller having an auto-calibration feedback path;

FIG. 8 illustrates a laser calibrator for adjusting a calibration factor in the laser controller;

FIG. 9 illustrates the display of the laser console during the auto-calibration routine;

FIG. 10A is a schematic diagram of a detector for use in the system of FIG. 2;

FIG. 10B is a perspective view of a detector;

FIG. 10C is a view of components in the interior of the detector of FIG. 10B; and

FIG. 11 is a flow chart of a method of calibrating the laser controller.

### DETAILED DESCRIPTION OF THE EMBODIMENTS

The prior art laser system illustrated in FIG. 1 is an example of a photo-coagulator laser system. Standard photo-coagulator laser systems include a photo-coagulator laser unit 10 followed by an optical delivery path. Upon exiting the laser unit 10, the laser beam travels through the optical delivery path, which prepares and delivers the laser beam to a delivery point at a distal end of the optical delivery path. During treatment the delivery point is applied to the patient's eye 100. The optical delivery system generally includes fibre optic cable 20, slit lamp adaptor 30, slit lamp microscope 40, beam splitter 50, and a delivery end (contact lens 60). The contact lens 60 (during treatment) usually contacts the area of the eye that requires treatment, and allows the laser beam to pass through to the eye. Other types of optical delivery path may be used, including an endo-ocular probe, a laser indirect opthalmoscope and a surgical microscope adapter.

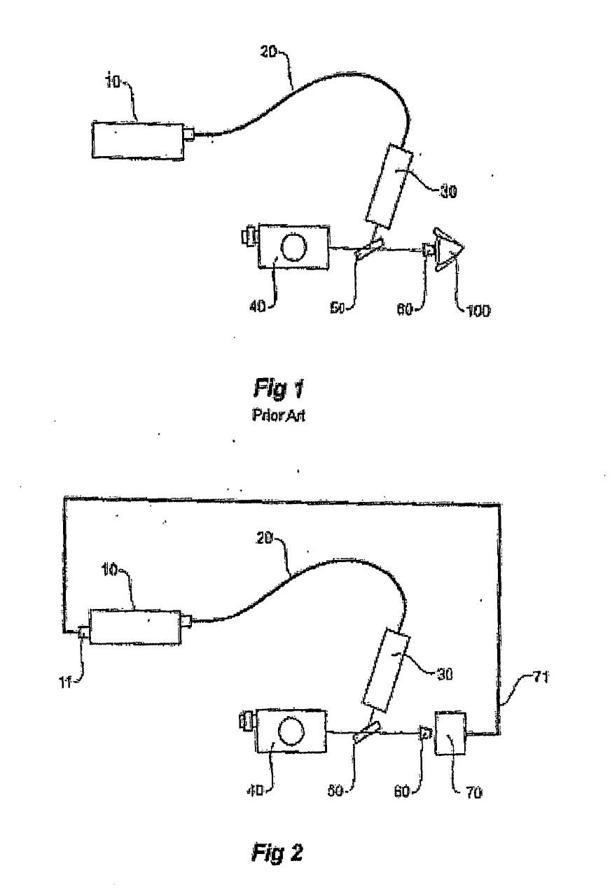

FIG. 2 shows an overview of a laser system incorporating the auto-calibration system described herein.

A detector 70 for measuring the power of the laser beam is located at the delivery point of the entire optical delivery path. This assists in giving a true measurement of the power of the beam that is actually delivered to the patient's eye. The measurement of the power of the beam made by the detector at the delivery end is then compared with the desired or required power level for delivery. As described in more detail below, this information is used to adjust a calibration factor that is used in controlling the power of the laser beam generated by laser console 10. Accordingly the power generation compensates for the effect of the optical delivery path.

This allows the power of the generated beam to be controlled to provide the desired laser power level to the patient, even though the optical delivery path may vary significantly for different procedures. The described auto-calibration also accounts for power deviations caused by component variation and degradation in the delivery path, as well as within the laser console itself.

The laser system calibration method is carried out at the practitioner's discretion, but preferably prior to use for each patient. In one arrangement the laser system locks to prevent more than ten procedures being performed without an auto-calibration. Once the laser system has been calibrated, the detector 70 is removed from the delivery point to allow treatment of the patient's eye 100.

Generally, deviations in transmission factors of the delivery system result in a loss of power of the laser beam, however if the laser system is calibrated to account for a loss and, for example the laser system components are cleaned or replaced at a later stage, then the power of the laser delivered at the delivery end can become greater than that calibrated for, resulting in possible injury to the patient.

As can be seen in FIG. 2, the system according to the present invention includes detector 70 which as previously described, is placed behind the contact lens 60 so as to measure the power of the laser beam at the end of the delivery path. Detector 70 converts the measure of the power of the laser beam to an electrical signal which is then fed via communication link 71 to an input 11 of laser console 10. This electrical signal is converted into a digital signal (unless the signal is already a digital signal) which is then provided to a processor in laser console 10. The processor then calculates the deviation of the power measured by detector 70 from the desired power at the delivery end and performs appropriate steps to compensate for differences between the desired and measured power. Due to this compensation, the ultimate power delivered to the patient during treatment is at or close to the desired power for the treatment.

### Description of the Laser Console

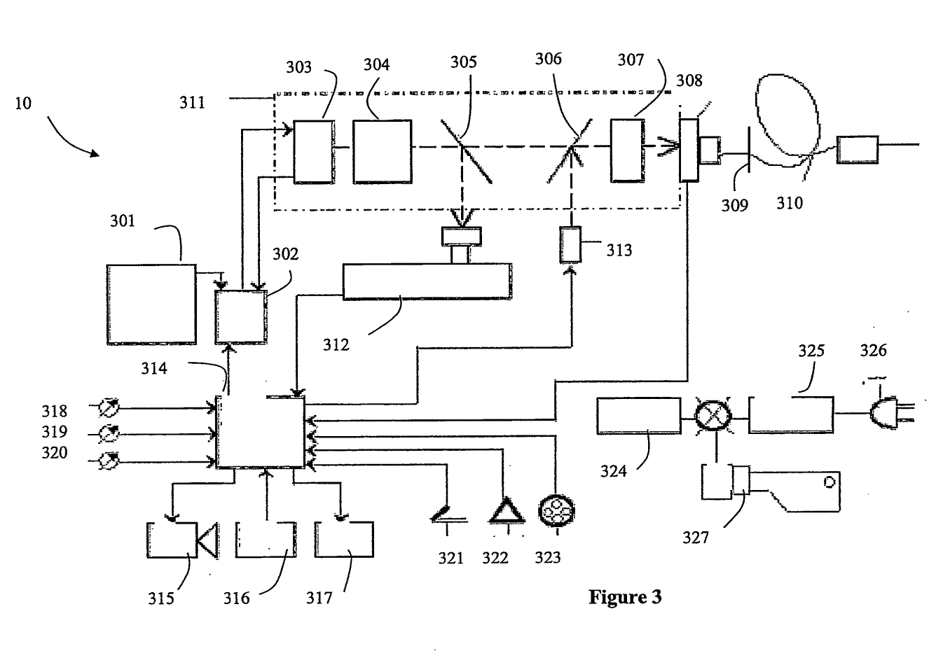

FIG. 3 shows the laser console 10 in greater detail. The main laser power supply 301 supplies the required current to produce the laser. The main laser power controller 302 is a module that controls the current to the main laser so that the output power is equivalent to the desired power. The laser diode 303 is used to generate the laser beam for the procedure. The wavelength of the laser is 810 nm, which is infrared and invisible to the human eye. The laser produced by diode 303 passes through the main laser collimator lens set 304, which shapes the laser beam so that the beam can be focused onto the fibre-optic cable.

After the lens set 304, the beam passes through beam splitter 305, which is a partially reflective mirror that splits the laser beam, providing a percentage of the laser beam to a photo sensor 312 that forms part of a safety system.

The part of the beam that is not diverted by the beam splitter 305 reaches the aiming beam combiner 306, which is a special mirror that combines the main laser beam from diode 303 with an aiming laser beam received from laser diode 313. The aiming laser beam has a visible beam (red) that is used by the physician to aim the laser. In one arrangement the aiming beam laser has a wavelength of 630 nm and a maximum power of 1 mW. In contrast, the main beam has a maximum power of 2.4 W.

After the aiming beam combiner 306, the combined beam passes through a fibre coupler lens set 307 that focuses the laser beam onto the fibre optic cable of the optical delivery path.

Laser cavity 311 is a metal box which contains the main laser diode 303, and the optical components 304, 305, 306 and 307 used to adjust the shape, focus and direction of the laser. The aiming laser diode 313 may also be included in the laser cavity. The optical delivery path 310 is connected to an output nozzle of the laser cavity 311. The cavity 311 is sealed to protect the optical system from dust and humidity. At the output nozzle of the laser cavity 311, there is an optically-coupled fibre lock sensor 308 that indicates to the controller whether there is a fibre optic cable connected to the laser console 10. A mechanical laser shutter 309 is connected by a hinge to the laser console 10 to cover the output nozzle when no delivery device is connected to the laser console 10.

The laser console 10 may be connected to an optical delivery path 310 which includes a fibre optic cable used to deliver the laser beam to the patient's eye. Examples of optical delivery paths include an endo-ocular probe, a slit lamp adaptor, a laser indirect opthalmoscope and a surgical microscope adapter.

Some of the beam split by beam splitter 305 is provided to the main laser safety photo-sensor 312, which is a photodiode that reads the power level and provides an electronic signal used to ensure safe laser operation.

Processor 314 controls the functioning of all the laser equipment, and is in electronic communication with most of the components of the laser console 10. In one arrangement the processor 314 includes a microprocessor from the 8032 family, flash memory, e2prom and a watchdog unit. A buzzer 315 connected to the processor 314 is used to generate alarms, beeps and other audible signals.

Keyboard 316 is used as an interface for the physician or operator to control the operating mode and parameters of the treatment, and the alphanumeric display 317 is used as an interface to show the treatment data and parameters to the physician using the laser console 10.

A laser power knob 318 is preferably a rotary knob allowing the physician to set the main laser power. The power knob includes an encoder from which output signals are read and interpreted by the processor 314 and displayed to the physician.

The pulse-duration-select dial button 319 is a rotary knob allowing the physician to set the duration of a laser shot. The button 319 includes an encoder from which output signals are read and interpreted by the processor 314 and displayed to the physician, for example, on display 317.

The pulse interval select dial button 320 is a rotary knob which allows the physician to set the repeat interval. Diode button 320 includes an encoder from which output signals are read and interpreted by the processor board 314.

Foot switch 321 is used to fire the laser beam. The foot pedal 321 is optically coupled to the laser console 10 to provide electrical safety.

Interlock unit 322 is an optional device for additional laser safety. The interlock input 322 allows a switch to be connected to the laser console 10 to disable the laser when an external door is opened inadvertently. If the user chooses not to use the remote interlock, then a by-pass connector must be inserted into the interlock unit 322 to enable operation of the laser.

The “autokey” connector 323 contains electrical circuitry used to provide information to the laser console 10 that indicates what optical delivery path has been connected to the laser console 10. Each optical delivery path 310 has different transmission properties which affect the laser power that reaches the patient's eye 100. Information provided to the laser console 10 via the autokey connector 323 enables the console 10 to recognise the delivery device in use so that the processor 314 can calculate a transmission factor (FAT) to compensate for the attenuation of laser power along the optical delivery path 310.

An electronic power supply 324 supplies the required power to the circuits of the power controller 302 and the processor board 314. EMI/EMC line filter 325 is a module that filters the electrical noise from the mains line to protect the laser from malfunction and damage due to possible power surges. Mains cable 326 connects the laser console 10 to an electric outlet. Switch 327 is an on/off switch allowing the user to turn the laser console 10 on or off.

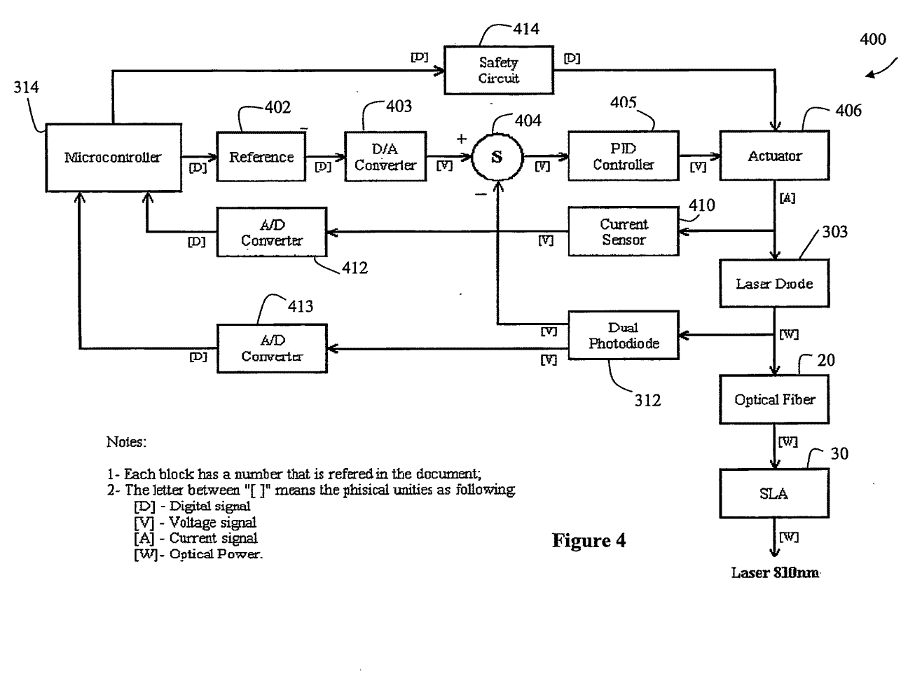

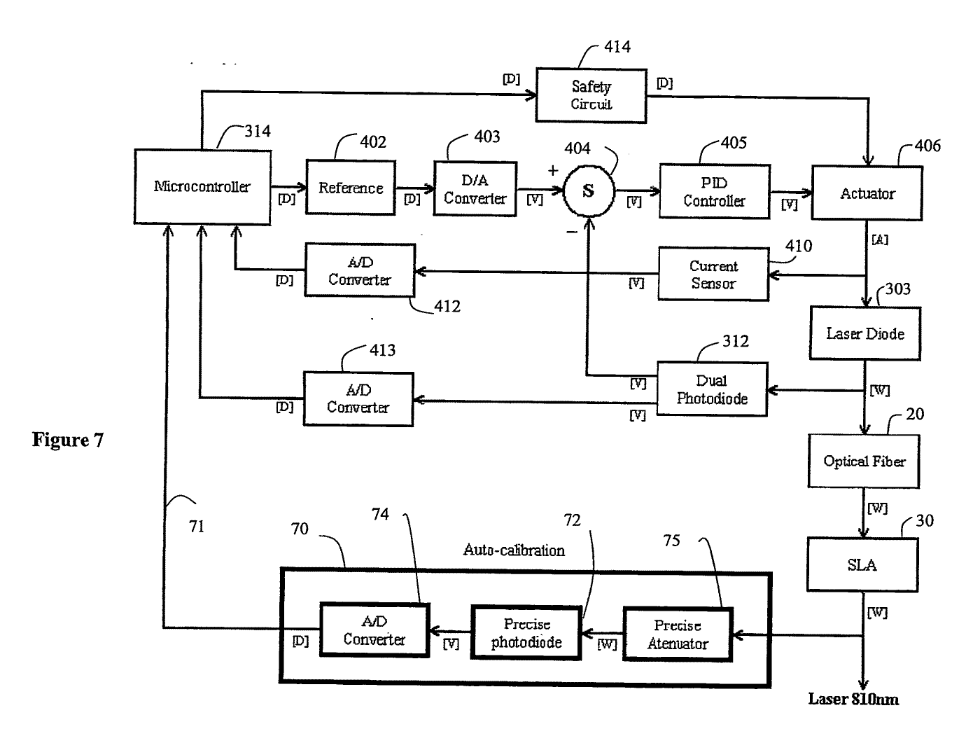

FIG. 4 shows a functional block diagram of a power control system 400 for use in the laser console 10. FIG. 4 shows the functional blocks without the auto-calibration functionality, which is shown in FIG. 7.

Microcontroller 314 controls a safety circuit 414 that sends a signal to actuator 406 to turn off the laser diode 303 if a fault is detected, thus preventing the laser from firing a shot if the power level is not in specified limits. The microcontroller 314 is a unit where operational software is stored and executed. When a command is received to activate the laser, a reference block 402 generates a reference signal that relates to the level of power desired at the delivery point at the end of the optical delivery path 310. The reference block 402 may be a module of the microprocessor 314.

The reference signal provided by reference block 402 is converted to an analogue voltage by the D/A converter 403. In turn the analogue signal is provided to subtraction block 404. The subtraction block 404 has another input signal that corresponds to the amount of power that the laser diode 303 emits in the laser cavity. The subtraction block 404 compares its two inputs to generate an error signal that is provided to PID controller 405. The input signal of the PID controller 405 is thus the difference between the desired power and the actual power generated in the laser cavity 311. The PID controller 405 amplifies the error signal, taking into account the dynamics of the system, and sends the amplified signal to the actuator 406 which directly controls the current to the laser diode 303.

As described above, the output of the laser diode 303 may be transmitted by an optical delivery path, for example, optical fibre 20 and slit lamp adaptor 30.

Dual photodiodes 312 monitor the output of the laser diode 303 to provide feedback signals for the power control and safety functions. One of the photodiodes 312 sends a voltage signal corresponding to the power level in the laser cavity 311 to the subtraction block 404. The other photodiode 312 sends a voltage signal to the A/D converter 413 which provides a digital signal to the microcontroller 314 indicative of the actual power level. The signal that passes via A/D converter 413 is not used in the power feedback loop but instead is used in the safety circuit 414. If the power in the laser cavity 311 exceeds the set power by more than 20%, the laser diode 303 is switched off immediately and an error message is displayed on the alphanumeric display 317.

When the laser beam is transmitted through the optical delivery path, the beam is attenuated and some laser power is lost in the transmission. It is necessary to estimate the attenuation of each optical delivery path and spot size and to use this information in the power control of the laser console 10. For example, if slit lamp adaptor 30 with a selected spot size of 200 micrometers has an estimated attenuation of 20%, the power generated in the laser cavity 311 must be increased by 20% so that the power that hits the patient's eye 100 matches the power set by the physician.

The amount of attenuation is noted at the factory during production of the optical delivery path. Based on the attenuation a correction factor is calculated, namely the transmission factor (FAT), also referred to as the compensation factor. The transmission factor is recorded in the memory of the laser console 10 for each type of delivery path and spot size for which the laser console 10 is used.

The following paragraphs describe how the transmission factor is used by the laser console 10 to adjust the power when using a slit lamp adaptor 30. The same system is used for other delivery devices, although endo-ocular probes and laser indirect opthalmoscopes have only one fixed spot size.

The slit lamp adaptor 30 used for the i-MP procedure has a magnification changer which produces five different laser spot sizes. In one arrangement the spot sizes are: 0.8 mm, 1.0 mm, 1.5 mm, 2.5 mm and 4.3 mm. These values represent the diameter of the laser beam at the focal point of the slit lamp adapter 30.

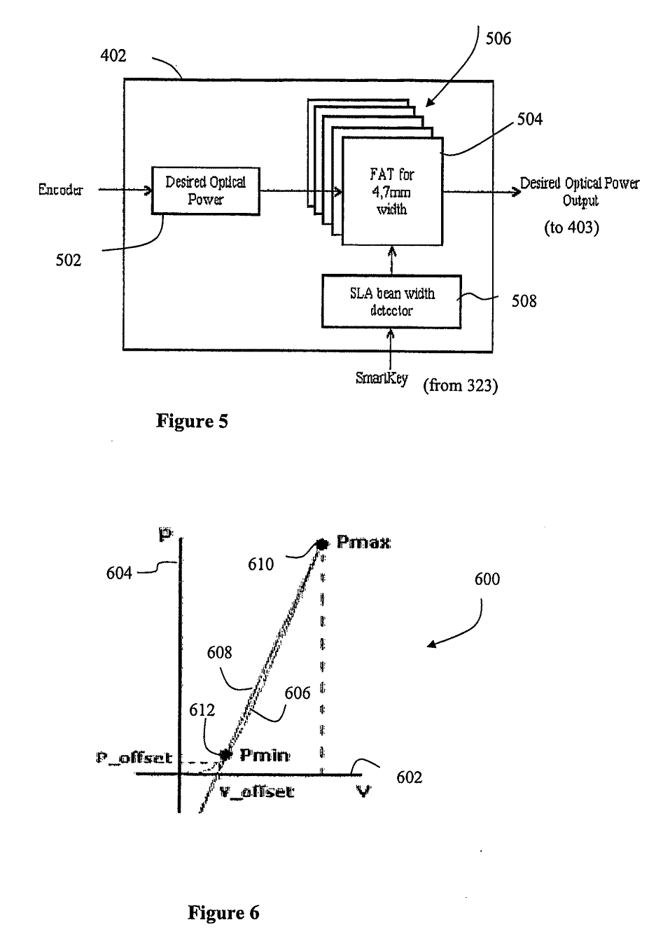

For each spot size selected, the laser beam passes through a different lens set. The attenuation of the beam is consequently different for each spot size. In order to compensate for the attenuation, the laser console 10 must be informed of the selected spot size so that the correct transmission factor (FAT) is used in the calculations. FIG. 5 illustrates the operation of reference block 402 for adjusting the power for different spot sizes. Reference block 402 may be implemented as a sub-system of the micro-controller 314.

Block 402 receives an input from the autokey 323 that enables slit-lamp-adaptor beam-width detector 508 to recognise the type of optical pathway in use and the selected spot size. Detector 508 is thus an optical path identifier. Block 402 uses this information to select an appropriate FAT (for example the FAT 504 for a spot width of 4.7 mm) from a set of transmission factors 506 stored in memory. Another input to block 402 enables the physician to specify the desired optical power, for example by means of power knob 318. Block 402 multiplies the desired optical power 502 by the selected FAT 504 to produce a desired optical power output, which is presented to the D/A converter 403.

As a result of this control system, the power delivered to the patient's eye 100 is theoretically equivalent to the power set by the physician for any power level. If the parameters of the PID controller 405 are well selected, there should be no oscillations of power generated by the laser diode 303. However, there are many factors that can affect the power delivered to the patient's eye which cannot be detected by the system shown in FIG. 5. The auto-calibration device shown in FIG. 7 was designed to address some of the limitations of the arrangement of FIG. 5, thereby to increase the precision of the power control.

One limit to the power control system is the non-linearity of the laser diode 303. FIG. 6 illustrates the power output of the laser diode 303 in response to a given reference voltage. The graph 600 shows power 604 versus voltage 602. The ideal response 608 of the photo diode 303 is linear between a minimum point 612 and a maximum point 610. In practice, the actual response code 606 is non-linear as shown in the FIG. 6.

Other errors in power transmission may be caused by the fibre optic coupling. The fibre optic couplings include high precision connectors where the physician or operator inserts the delivery devices via the optic cable into a receiving port and twists the fibre optic clockwise until the end connector reaches the end of the course. However, a small shift in position can be caused by simply removing the fibre optic cable and inserting it back into the port. This can cause an error of up to 5% in the transmitted power. Since the power controller 400 operates within the laser cavity 311, this power error is not detected by the system and is therefore not corrected for.

In addition, any type of dust or dirt on the lenses of the slit lamp adapter 30 can cause attenuation in the power delivered by the system. Again, because this happens outside the laser cavity 311, the error is not corrected by the system of FIG. 4.

Aging of the laser diode 303 is a major factor causing error in the power control system. During factory calibration the laser diode 303 presents a characteristic curve, for example that shown in FIG. 6. The system is then calibrated between the minimum point 612 and maximum point 610 so that within the dynamic range of the laser diode 303 the error is the smallest possible. As the diode 303 ages, the shape of the curve changes and consequently the minimum and maximum points may shift. If a laser diode 303 is only calibrated during production, this error tends to increase over time as the laser is used.

Other factors contributing to error in the system include the appearance of microfissures in the fibre optic cable or a misalignment of the fibre coupling.

### Auto-Calibration System

The power control system using the auto-calibration facility adds a further compensation factor (ACFAT), which is generated by the auto-calibration system. The desired power selected by the physician is multiplied by the FAT and by the ACFAT to generate the reference voltage presented to the D/A converter 403.

The functional block diagram shown in FIG. 7 is similar to the functional block diagram of FIG. 4, with the addition of detector 70 that reads the laser power at the delivery point of the optical delivery path and provides an electrical signal indicative of the laser power back to the microcontroller 314. The detector 70 includes a precise optical attenuator 75, a photodiode 72 to measure the incident power, and an A/D converter 74 to provide a digital signal that may be fed back to microcontroller 314 via communication link 71. The attenuator 75 attenuates the incident laser power (which may, for example be 1 W, for some procedures) to the operational range of the photodiode, so that the photodiode 72 does not saturate or get damaged.

For each power range to which the power of the laser console 10 can be set, there is a specific ACFAT that is calculated every time an auto-calibration routine is executed. When auto-calibration is completed, the laser control system returns to its normal operating mode as illustrated in FIG. 4.

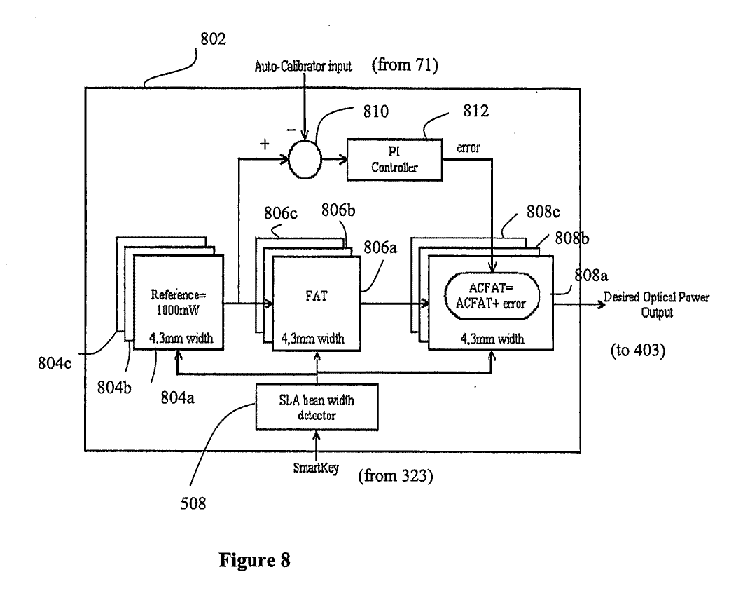

Auto-calibration using laser calibrator 802 is illustrated in FIG. 8. The laser calibrator 802 replaces block 402 in the functional block diagram of FIG. 7.

As before, the calibrator 802 receives an input from the autokey 323 which enables the beam-width detector 508 to detect which optical delivery path and spot size have been selected. Dependent on the selected spot size, the laser calibrator 802 retrieves a FAT 806a-c corresponding to the selected spot size. The calibrator also selects an ACFAT from the set 808a-c corresponding to the selected spot size. Furthermore, the laser calibrator 802 selects a reference input 804a-c corresponding to the selected spot dimensions. For example, for a spot width of 4.3, the reference input to be used in the calibration procedure is 1000 mW.

The ACFAT 808a is initialised to a value of 1.0. The laser is then fired (by operating foot switch 321) and the detector 70 reads the received power at the delivery point and sends the information to the microcontroller 314. Subtracter block 810 compares the received measurements to the selected reference 804a and generates an error signal. The error signal is provided to PI controller 812 and the output of PI controller 812 is added to the ACFAT 808a. Note that if the measured power is the same as the reference power, then the error signal is 0 and there is no adjustment to the ACFAT 808a. If the power at the output of the slit lamp adaptor 30 is less than the reference power, a positive value that is proportional to the error and the dynamic response of the external control loop is added to the ACFAT. Conversely, if the measured power is greater than the reference, a negative value is added to the ACFAT 808a. After a number of iterations, the ACFAT 808a converges to a fixed value, thereby causing the reference signal and the measured power to approach equality.

According to the i-MP protocol, the power levels are proportional to the laser spot size and there is a need to ensure that the delivered power deviates by less than 5%.

According to the i-MP protocol, the power at the delivery end of the slit lamp adaptor 30 is dependent on 3 major factors, namely the greatest linear dimension of the lesion, the weight of the patient and the skin pigmentation level of the patient. In order to minimise the error due to the non-linearity of the laser diode 303, three reference points are used and the auto-calibration device reads the actual delivered power at each of these 3 points:
lesion size smaller than 1.5 mm, use laser spot size of 1.5 mm;
lesion size between 1.5 mm and 3.0 mm, use laser spot size of 2.5 mm; and
lesion size greater than 3.0 mm, use laser spot size of 4.3 mm.

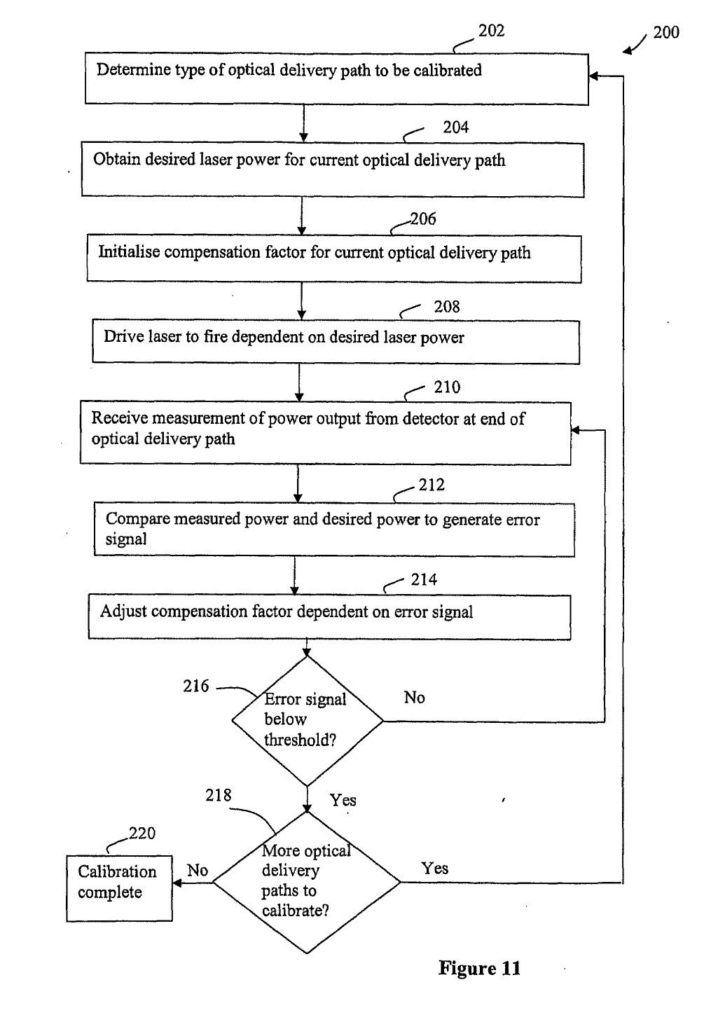

FIG. 11 illustrates the method followed for the auto-calibration. In step 202 the type of optical delivery path to be calibrated is determined. This determination may be dependent on the output of the beam-width detector 508. In step 204, a desired laser power is obtained corresponding to the current optical delivery path.

In step 206 the compensation factor ACFAT is initialised to 1.0 for the current optical delivery path. Then, in step 208 the laser controller 400 drives the laser to fire dependent on the desired laser power. In step 210 the detector 70 measures the power output at the end of the optical delivery path and provides the measured power to the microcontroller 314. Then, in step 212 the subtraction block 810 compares the measured power and the desired power to generate an error signal. The PI controller 812 in step 214 adjusts the ACFAT so as to reduce the error signal.

Step 216 checks whether the error signal is below a threshold value. If the error signal is still too high (the No option of step 216) then the control flow returns to step 210 to continue the auto-calibration. If the error signal is sufficiently small (the Yes option of step 216) then the ACFAT has been determined for the current optical delivery path and in step 218 the laser calibrator 802 checks whether there are more optical delivery paths to calibrate (i.e. whether the other spot sizes have yet been calibrated). If there are no more spot sizes then the calibration is complete (step 220). Otherwise, process flow returns to step 202 to perform the auto-calibration for the other delivery paths.

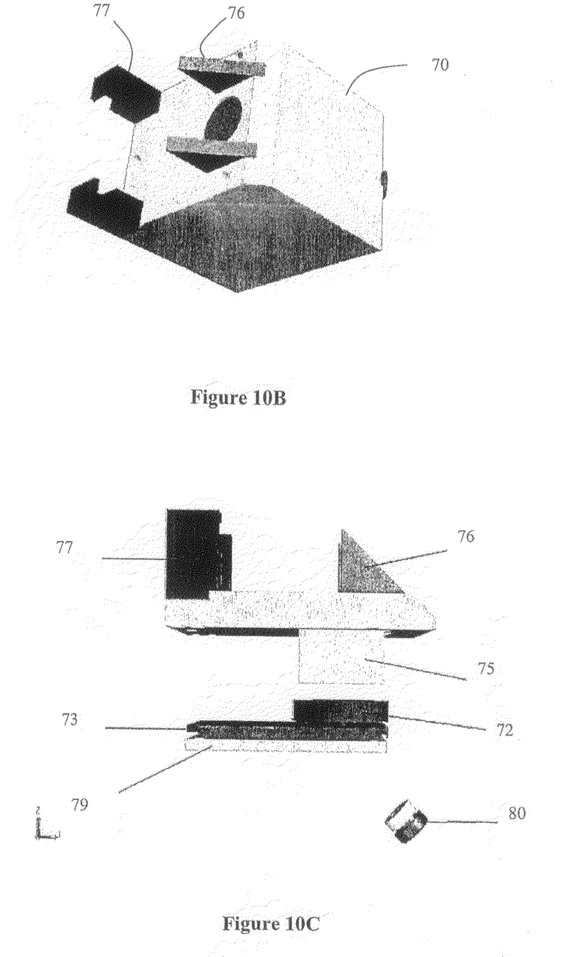

As can be seen in FIG. 10A, detector 70 contains a high-precision photodiode 72, which converts the power measurement of the laser received by photodiode 72 into an electrical signal which is then amplified by amplifier 73 and then converted into a digital signal by ADC 74. This signal is then transmitted into laser console 10 via cable 71, which attaches to console 10 via input port 11. Of course, it will be understood that the ADC conversion may alternatively be performed within laser console 10 itself, or any other convenient location. Sensor 70 has an optical filter 75 which blocks light of visible wave-length allowing only the near infrared wave-length to reach the photodiode 72. The filter 75 attenuates the laser power to prevent saturation of the photodiode 72.

FIG. 10B shows a particular detector 70 designed to be attached to a slit lamp adaptor through a mechanical coupling system 76, 77. Detector 70 and the body of the slit lamp adaptor 30 have marks to guide the appropriate positioning of the detector 70.

Before commencing the auto-calibration, the physician or operator attaches the detector 70 to the body of the slit lamp adaptor. The mounting posts 76 and guiding posts 77 assist the operator in attaching the detector 70 onto the slit lamp adaptor body. As shown in FIG. 10C, the components of the detector 70 also include laser beam attenuation filter 75, photodiode electronics circuit board 73, a precision photodiode 72, electronic circuit board fixation mounts 79 and a power supply and signal cables protecting boot 80.

In one arrangement, the adjusting margin of the auto-calibration is approximately 3% of the factory setting. This prevents the use of the equipment out of the calibrated and nominal operational conditions. In one arrangement the system permits 10 treatments to be performed between calibrations. If the number of treatments exceeds 10, the laser console will self-lock and display a message to the physician requiring calibration, preventing the physician from performing a new treatment until a new auto-calibration has been successfully performed. It is recommended that an auto-calibration be performed every time the laser console 10 is not used for a period of 3-5 days, or when the delivery device has been disconnected.

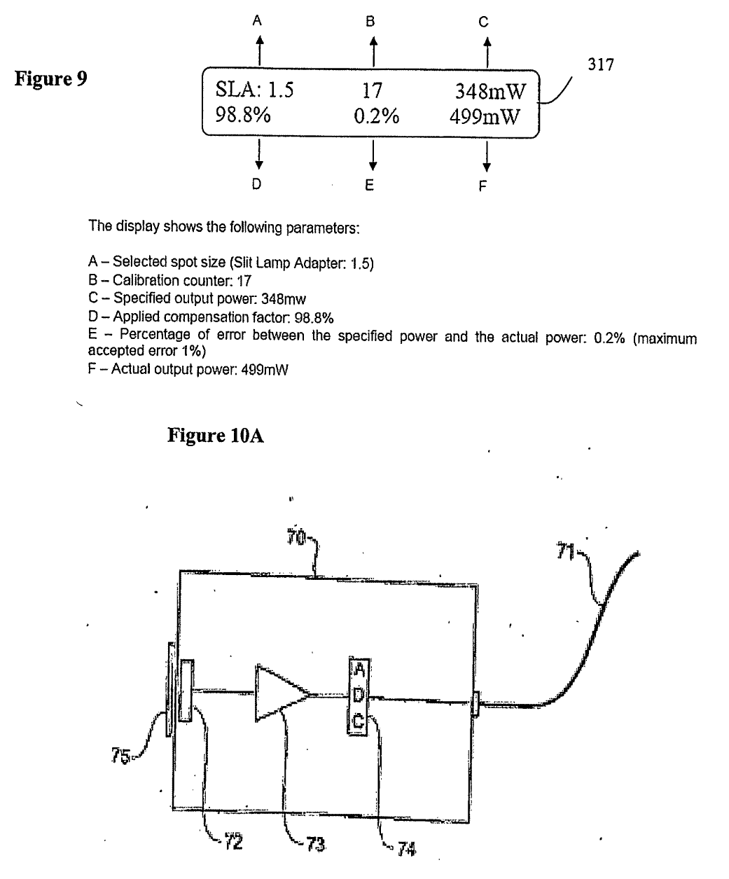

The operator presses a mode button until the display 317 shows the message “auto-calibration mode”. The operator presses a select/okay button to enter this mode, following which the operator is prompted to confirm whether he or she wishes to enter the auto-calibration procedure. After confirmation a message will prompt the user to wear safety goggles and confirm that goggles have been put on. The user then positions the detector 70 on the slit lamp adaptor 30. The aiming laser may be automatically turned on to assist the user in positioning the detector 70. The next message displayed will ask the user if the marks at the detector 70 and slit lamp adaptor 30 are aligned. The operator must press a select/okay button to confirm. At this point the procedure requires the user to select a spot size, for example 1.5 mm, and the system will display a message confirming when the thumb wheel setting the spot size is at the correct position. The system then prompts the user to fire the laser by pressing the foot pedal 321. As soon as the foot pedal is pressed the display shows calibration parameters to allow the user to monitor the calibration. An example is shown in FIG. 9. Here the display 317 indicates a selected spot size of 1.5 mm and a specified output power of 348 mW. The actual output power is also displayed, together with the percentage of the error between the specified power and the actual power. In the example the percentage error is 0.2%. The threshold defining the maximum accepted error is 1%. A calibration counter counts down the time taken in the calibration.

If during calibration the error exceeds 0.5% or the foot pedal 321 is released, the calibration counter restarts counting. A maximum time allowed for calibration of each spot size is 120 seconds. If the laser calibrator 802 cannot calibrate the output power within this time limit, an error message will appear on their display 317 and the auto-calibration procedure is aborted.

Whilst the foot pedal 321 is kept pressed, the laser is activated and an audible beep will be sounded continuously by buzzer 315. When the calibration counter reaches 0, the laser is turned off and the display prompts the user to shift the spot size to 2.5 mm. The system then repeats the procedure for the 2.5 mm and 4.3 mm spot sizes. Once the 4.3 mm calibration step is finished, the display 317 shows a message confirming the successful completion of the auto-calibration.

To perform the procedure, the user will connect the sensor 70 to the laser console 10 via input port 11 and electric cable connector 71. The operator will then activate the self-calibration routine from the menu.

The display menu will then prompt the operator to follow an automatic procedure in which he or she will:

select a 1.5 mm spot size of the laser on the slit lamp adaptor 30 and fire the laser;

switch to a 2.5 mm spot size and fire again;

switch to a 4.3 mm spot size and fire one more time.

Each time the laser is fired, the auto-calibration device measures the output power detected by the photodiode and feeds it back into the laser console 10. The operational software then compares the power at the end of the delivery device with the power delivered by the laser cavity and feeds back the difference into the operational software through a Proportional Integral (PI) controller. The resulting digital power difference signal is used to calculate a new compensation factor for the slit lamp adaptor, which will compensate for all losses in the optical path from the laser cavity to the patient's eye. It will be understood that a Proportional Integral Derivative (PID) controller may also be used

As an example, if the slit lamp adaptor's determined transmission factor (FAT) is calculated at 75% for a particular spot size, the maximum output power at the patient's eye of a 2.5 W laser console will be 1875 mW and therefore, this will be the maximum power the user will be able to adjust the laser to when using the slit lamp adaptor as a delivery device for that particular spot size. On top of this gross transmission factor comes the determined compensation factor (ACFAT) from the auto calibration device, which can add up to 20% power compensation. If in the above example, an auto calibration routine is performed and the determined compensation factor is calculated at 10%, the maximum adjustable power will be 1687 mW.

It will be appreciated by those skilled in the art, that the embodiment provides a laser system which can provide an improved accuracy in the desired power of the laser delivered to the patient's eye. The invention is not restricted in its use to this particular application. The invention may also be applied to other retinal laser systems such as photo-coagulator systems and photodynamic therapy lasers. Neither is the present invention restricted in its preferred embodiment with regard to the particular elements and/or features described or depicted herein. It will be appreciated that various modifications can be made without departing from the principles of the invention. For example, the measurement taken by the detector could be converted into a digital signal suitable for wireless transmission to laser console 10. Furthermore, the CPU containing the operational software could be provided separately from the laser console, and control appropriate control functions remotely. Furthermore, while the power of the laser beam delivered to the patient's eye may be controlled by a suitable component within the delivery path, the power could equally be controlled by modifying the operation of the laser console itself. Therefore, the invention should be understood to include all such modifications within its scope.

It will be understood that the invention disclosed and defined in this specification extends to all alternative combinations of two or more of the individual features mentioned or evident from the text or drawings. All of these different combinations constitute various alternative aspects of the invention.

1. A method of calibrating a laser system in which a laser unit is operable to fire a laser beam that is guided along an optical delivery path to a delivery point at a distal end of the optical delivery path, the method comprising:
defining a desired laser power;
initialising a compensation factor for the optical delivery path;
driving the laser unit to fire a laser beam dependent on the desired laser power and the compensation factor;
receiving a measurement signal related to laser power at the delivery point;
comparing the measurement signal and the desired laser power to generate an error signal; and
adjusting the compensation factor dependent on the error signal.

2. A method according to claim 1 further comprising: identifying the optical delivery path.

3. A method according to claim 1 wherein the laser beam has a selectable spot size at the delivery point, the method further comprising the step of identifying a selected spot size, and wherein said defining step retrieves a desired laser power associated with the selected spot size.

4. A method according to claim 1 wherein said initialising step comprises retrieving a predefined attenuation factor associated with the optical delivery path.

5. A method according to claim 4 wherein the predefined attenuation factor for the optical delivery path is multiplied by an auto-calibration factor and wherein said adjusting step adjusts the auto-calibration factor dependent on the error signal.

6. A method according to claim 1 wherein the method is repeatedly applied to calibrate the laser system for a plurality of optical delivery paths.

7. A method according to claim 3 wherein the method is repeatedly applied to calibrate the laser system for a plurality of laser spot sizes.

8. A laser calibration system for a laser unit operable to fire a laser beam that is guided along an optical delivery path to a delivery point at a distal end of the optical delivery path, said laser calibration system comprising:
a laser controller operable to drive the laser unit to fire the laser beam dependent on a desired laser power and a compensation factor associated with the optical delivery path;
a detector operable to generate a measurement signal related to laser power at the delivery point; and
a laser calibrator adapted to generate an error signal dependent on a comparison of the desired laser power and the measurement signal and to adjust the compensation factor dependent on the error signal.

9. A laser calibration system according to claim 8, further comprising: a path identifier for identifying the optical delivery path.

10. A laser calibration system according to claim 9 wherein said path identifier identifies an optical delivery path selected from the group consisting of
a) a slit lamp adapter, b) an endo-ocular probe, c) a laser indirect opthalmoscope and d) a surgical microscope adapter.

11. A laser calibration system according to claim 9 wherein said path identifier further identifies a spot size selected for the optical delivery path.

12. A laser calibration system according to claim 8 further comprising data storage that stores a set of predefined attenuation factors corresponding to one or more optical delivery paths.

13. A laser calibration system according to claim 12 wherein said laser calibrator is operable to adjust an auto-calibration factor that is multiplied with the predefined attenuation factor for the optical delivery path.

14. A laser calibration system according to claim 8 wherein said detector comprises a photodiode that generates a signal related to laser power incident on the photodiode.

15. A laser calibration system according to claim 14 wherein said detector further comprises an optical attenuator to attenuate the laser power incident on the photodiode.

16. A laser calibration system according to claim 8 wherein said detector comprises positioning means to position said detector on a component of the optical delivery path such that the laser beam emitted at the delivery point is incident on the photodiode.

17. A laser calibration system according to claim 16 wherein said positioning means comprise one or more guiding posts extending from a body of said detector.

18. A laser calibration system according to claim 16 wherein the component of the optical delivery path is a slit lamp adapter.

19. A laser calibration system according to claim 8 further comprising a display for displaying information related to the calibration of said laser system.

20. A laser calibration system according to claim 8 wherein said laser calibrator comprises a proportional integral (PI) controller that processes the error signal.

21. A laser system including:
a laser for generating a beam of laser light;
an optical delivery path provided by at least one selected component for a given procedure;
a detector for placement at the end of the optical delivery path for measuring the power of the laser beam at the end of the delivery path; and
laser power modification means for modifying the power of the laser beam in accordance with the measurement obtained by the detector.

22. A computer program product comprising machine-readable program code recorded on a machine-readable recording medium, for controlling the operation of a data processing apparatus on which the program code executes to perform a method calibrating a laser system in which a laser unit is operable to fire a laser beam that is guided along an optical delivery path to a delivery point at a distal end of the optical delivery path, the method comprising:
defining a desired laser power for the optical delivery path;
initialising a calibration factor for the optical delivery path;
driving the laser unit to fire a laser beam dependent on the desired laser power and the calibration factor;
receiving a measurement signal related to laser power at the delivery point;
comparing the measurement signal and the desired laser power to generate an error signal; and
adjusting the calibration factor dependent in the error signal.

23. A computer program comprising machine-readable code for controlling the operation of a data processing apparatus on which the program code executes to perform a method of calibrating a laser system in which a laser unit is operable to fire a laser beam that is guided along an optical delivery path to a delivery point at a distal end of the optical delivery path, the method comprising:
defining a desired laser power for the optical delivery path;
initialising a calibration factor for the optical delivery path;
driving the laser unit to fire a laser beam dependent on the desired laser power and the calibration factor;
receiving a measurement signal related to laser power at the delivery point;
comparing the measurement signal and the desired laser power to generate an error signal; and
adjusting the calibration factor dependent in the error signal.

24. (canceled)

25. (canceled)
